(19) United States
(12) Patent Application Publication
(10) Pub. No.: US 2012/0309864 A1
(43) Pub. Date: Dec. 6, 2012

(54) DENTURE BASE MATERIAL THAT IS FRACTURE-RESISTANT AFTER CURING AND IS OBTAINED FROM AUTOPOLYMERIZING OR COLD-POLYMERIZING COMPOSITIONS

(75) Inventors: Klaus RUPPERT, Maintal (DE); Kevin KERSCHER, Neu Anspach (DE)

(73) Assignee: HERAEUS KULZER GMBH, Hanau (DE)

(21) Appl. No.: 13/470,828

(22) Filed: May 14, 2012

(30) Foreign Application Priority Data
May 30, 2011 (DE) 10 2011 106 816.7

(51) Int. Cl. A61K 6/09; C08L 75/06; C08G 18/00

(52) U.S. Cl. 523/115; 525/256

(57) ABSTRACT

A fracture-resistant denture base material is obtained from an auto-polymerising or cold-polymerising composition containing

(A) a liquid monomer component;

(B) a powder-shaped component, and

(C) at least one initiator or one initiator system for auto- or cold-polymerisation,

whereby at least one member of the groups of aliphatic urethane acrylates and aliphatic urethane methacrylates is present in (A) or/and (B);

whereby the fracture-resistant material shows, in particular, a fracture toughness of >1.8 MPa*m^{1/2} and a fracture work of >400 J/m² after curing, and, preferably also shows a difference in the yellow value, also called b value, of maximally one unit after 8 weeks of storage in fully desalted water at 50° C. Preferably, no synthetic rubber is present.

The invention relates to denture base material that is fracture-resistant after curing and is obtained from auto-polymerising or cold-polymerising compositions.

### BACKGROUND

Cured denture materials tend to chip in thin regions upon removal from the investment material mould. Moreover, due to the limited motor skills of denture wearers, who are usually more advanced in age, dentures occasionally are dropped onto a hard surface (tiles, washbasin) which may also cause them to chip. Said chipping is associated with additional work and costs for the dental laboratory and is therefore undesirable.

For these reasons, there is a need in the market to have a denture material that tolerates said brief introduction of energy without the material getting damaged, so-called high impact materials.

Products meeting these requirements have been on the market for some time, but are all members of the group of hot-curing denture materials.

Currently, various technologies are in use in order to attain high fracture resistance, for example the ones described in EP1923037A2, namely

i. core-shell particles and
ii. special plasticising molecules, such as, e.g., butadiene copolymers or butadiene-acrylonitrile copolymers.

Referring to i): Due to the short swelling time of the beads required of auto- or cold-polymers, core-shell particles are usually unsuitable for attaining high fracture resistance. The available time is insufficient to allow the pearls to swell sufficiently.

Referring to ii): One problem in this regard is that many plasticising synthetic rubber molecules contain nitrile groups which tend to show yellowing, typically in the presence of oxidising substances such as, e.g. peroxides or atmospheric oxygen.

The invention is to specify a cold-polymerising denture base material that is fracture-resistant after curing. Compared to a reference material without additive, the cured material should show increased fracture toughness of >1.8 MPa*m^{1/2} and increased fracture work of >400 J/m².

Moreover, it should also be colour-fast to the extent possible, in that it yellows not at all or only little. In other words, it should remain virtually colourless and thus be colour-fast, whereby the extent of colour-fastness should be equivalent to a change of the yellow value by one unit only upon storage of the material in fully-desalted water for 8 weeks at 50° C. Another goal to pursue is that the results of the so-called sun test according to ISO 20795 should not be affected adversely by novel additives.

### SUMMARY OF THE INVENTION

The object is met according to the invention by fracture-resistant denture materials and/or denture base material that is fracture-resistant after curing, which are obtained from autopolymerising or cold-polymerising compositions containing:

(A) a liquid monomer component;
(B) a powder-shaped component which preferably comprises one or more bead polymer(s)
(C) at least one initiator for auto- or cold-polymerisation;
whereby, according to the invention, at least one member of the groups of aliphatic urethane acrylates and aliphatic urethane methacrylates is/are present in (A) or/and (B), in particular liquid aliphatic urethane diacrylate resin consisting of aliphatic urethane and acrylic segments is present in component (A). Preferably, the compositions are free of plasticising synthetic rubber.

The resulting material parameters after curing are a fracture toughness of >1.8 MPa*m^{1/2} and fracture work of >400 J/m². It is particularly preferred for the cured material to show an increase in its yellow value (b value) of maximally one unit after 8 weeks of storage at 50° C. in fully-desalted water.

### DETAILED DESCRIPTION

Obviously, reference in this description being made to denture material and denture base material is to be understood to refer to the starting composition prior to curing or the cured material and its properties, whichever it may be in the specific case.

In the scope of the invention, aliphatic urethane (meth)acrylates shall be understood to preferably be polymers with aliphatic urethane segments and (meth)acrylate segments in the polymer structure. The extent of the actual beneficial influence of these on the properties of the denture materials was unexpected. It is feasible, in particular, to dispense with synthetic rubbers altogether.

Urethane acrylates and urethane methacrylates are usually colourless, colour-fast, transparent, lack inherent odour, and do not impair the properties of the base formulation. The invention therefore also relates to the use of an aliphatic urethane acrylate or urethane methacrylate, in particular, e.g., Albiflex® XP 6/1166 made by Nano Resins (Geesthacht, Germany), as a modifying agent in auto- or cold-polymerising denture materials.

According to the technical specifications sheet of the company mentioned above of January 2010, Albiflex® XP 6/1166 is the following experimental product:

“Albiflex XP 6/1166 is a novel aliphatic urethane diacrylate resin intended for formulating high-performance elastomers. Albiflex XP 6/1166 is liquid at ambient temperature and can be cured by all radical-generating photoinitiators as well as by peroxides. It can be processed through all common casting or coating techniques. Composed of aliphatic urethane and acrylic segments, the special-adjusted structure of Albiflex XP 6/1166 provides an excellent combination of properties:
much higher strength than conventional diacrylates;
excellent adhesion to most plastic substrates;
better weather-resistance than polyester acrylates;
excellent compatibility with Nanocryl® products;
very low halogen content.

Combining aliphatic urethane and acrylic segments in the polymer structure generates an elastomeric material that joins the excellent chemical properties of acrylates and the high mechanical strength of aliphatic urethane resins.

Applications:

Albiflex XP 6/1166 is particularly well-suited for use in flexible coatings and sealing formulations in electrical/electronic applications. Acrylic adhesives can be optimised with regard to flexibility without deteriorating their strength and other properties.

Formulations and Processing:

Albiflex XP 6/1166 can be cured by virtually all radical-generating photoinitiators and peroxides. Albiflex XP 6/1166 can also be cured by light-sensitive pre-adducts. Albiflex XP 6/1166 shows good compatibility with aliphatic urethane (meth)acrylates, aromatic urethane (meth)acrylates, polyester (meth)acrylate resins, epoxy (meth)acrylates, amine (meth)acrylates, and melamine (meth)acrylates. Albiflex XP 6/1166 can be formulated together with acrylate monomers, acrylate oligomers, solvents, etc., for further modificat of the properties of the finished products. Further information regarding formulations can be furnished upon request. Formulation ingredients showing increased turbidity after being mixed with Albiflex XP 6/1166 should be avoided, since this is an indication of incompatibility. A colour change to white during curing, though, is not evidence of difficulties, but rather is an indication of a change of the refractive index.

Storage:

Albiflex XP 6/1166 should be stored in a cool and dry place. Exposure to direct sunlight should be avoided during storage.

Technical Specifications:

Appearance:

Clear liquid, Density at 20° C. [g/ml]: ˜1.0; Viscosity at 25° C. [mPa·s]: 30,000-60,000; Shelf life unopened 6 months.

According to the invention, the aliphatic urethane methacrylate is not to be, in particular, the known dental monomer UDMA (an addition product made from 2-hydroxyethylmethacrylate and 2,2,4-hexamethylenediisocyanate). This [substance] can be present in the compositions according to the invention serving its usual property as a further monomer, in particular as a cross-linker.

The fraction of aliphatic urethane (meth)acrylate in the compositions according to the invention is, for example, 0.1 to 20% by weight, preferably 0.1 to 10% by weight. The percentage fractions given refer to the total composition. The aliphatic urethane (meth)acrylate can be present either in the liquid monomer component (A) or in the solid component and/or powder component (B) or in both. Preferably, it is present in component (A).

In addition, the cold- or auto-polymerising denture materials according to the invention can contain one or more substance(s) from the groups of further monomers, filling agents, pigments, stabilisers, modifiers, antimicrobial additives, UV-absorbing agents, thixotroping agents, catalysts, and cross-linkers.

The monomers that are common in the field of dentistry are conceivable as further monomers:

Examples include polymerisable m monofunctional monomers for radical polymerisation such as mono(meth)acrylates, methyl-, ethyl-, butyl-, benzyl-, furfuryl- or phenyl(meth)acrylate, di- and/or polyfunctional monomers such as di- or polyfunctional acrylates and/or methacrylates, e.g. bisphenol-A-di(meth)acrylate, bis-GMA (an addition product made from methacrylic acid and bisphenol-A-diglycidyl-ether), di-, tri- or tetraethylene glycoldi(meth)acrylate, decandioldi(meth)acrylate, dodecandiol-di(meth)acrylate, hexyldecandiol-di(meth)acrylate, trimethylolpropan-tri(meth)acrylate, pentaerythritol-tetra(meth)acrylate as well as butandiol-di(meth)acrylate, ethyleneglycol-di(meth)acrylate, polyethyleneglycol-di(meth)acrylates, ethoxylated/propoxylated bisphenol-A-di(meth)acrylates.

Conceivable filling agents are e.g. pyrogenic or precipitated silicic acids, dental glasses such as aluminosilicate glasses or fluoroaluminosilicate glasses, strontium silicate, strontium borosilicate, lithium silicate, lithiumaluminiumsilicate, phyllosilicates, zeoliths, amorphous spherical filling agents based on oxides or mixed oxides (SiO₂, ZrO₂ and/or TiO₂), metal oxides with a primary particle size of approx. 40 to 300 nm, splitter polymers with a particle size of 10-100 μm (cf. R. Janda, Kunststoffverbundsysteme, VCH Verlagsgesellschaft, Weinheim, 1990, page 225 pp.) or mixtures thereof. Moreover, reinforcing agents such as glass fibres, polyamide or carbon fibres can be worked in.

The amount of filling agents that is usually used is 0 to 10% by weight, preferably 0 to 3% by weight, relative to the total denture plastic composition and/or the sum of components (A) and (B).

Suitable stabilisers include, e.g., hydroquinone monomethylether or 2,6-di-tert.-butyl-4-methylphenol (BHT).

Moreover, the denture base materials according to the invention can contain common additives, e.g. from the groups of antimicrobial additives, UV-absorbing agents, thixotroping agents, catalysts, and cross-linkers.

Rather small amounts of said additives—and of pigments, stabilisers, and modifiers—are used, e.g. a total of 0.01 to 3.0% by weight, in particular 0.01 to 1.0% by weight, relative to the total mass of the material.

The powder component of the two-component denture base material usually contains a polymer powder, in particular based on methacrylate, and/or a bead polymer based on methacrylate. In this field, bead polymers are often referred to as powder.

Bead polymers, in particular those made of (meth)acrylates, are known to the person skilled in the art. Polyalkyl(meth)acrylate-based bead polymers are obtained in known manner through precipitation polymerisation or suspension polymerisation. In this context, suspension polymerisation usually yields larger particles. The average particle sizes vary over a wide range and can, for example be from 0.1 μm to 250 mm. Cross-linked bead polymers are conceivable as well. Suitable multifunctional cross-linker molecules are evident from the listing of common monomers that are common in dentistry provided above.

Further comonomers can also be polymerised into the bead polymer. Exemplary monofunctional comonomers are styrene, alpha-methylstyrene, vinyltoluene, substituted vinyltoluenes such as vinylbenzylchlorides, butadiene, isobutylene, 2-chlorobutadiene, 2-methylbutadiene, vinylpyridine, cyclopentene, as well as (meth)acrylic acid esters such as methylmethacrylate, butylmethacrylate, butylacrylate, and hydroxyethylmethacrylate, as well as acrylonitrile, vinylacetate and vinylpropionate as well as mixtures of said comonomers. Preferred comonomers include vinylhalogenides such as vinylchloride, vinylesters such as vinylacetate, heterocyclic vinyl compounds such as 2-vinylpyridine, maleic acid and maleic acid derivatives such as, for example, maleic acid anhydride, fumaric acid and fumaric acid derivatives such as fumaric acid esters, acrylic acid, methacrylic acid as well as aryl(meth)acrylates such as benzylmethacrylate or phenylmethacrylate.

In a preferred embodiment, cross-linkers have been polymerised, at least in part, into the beads of the first (co)polymer and/or the beads of the second (co)polymer. Accordingly, the first and second bead polymers also comprise cross-linked and partly cross-linked bead polymers.

For cross-linking, it is common to resort to multifunctional comonomers or multifunctional oligomers. Aside from di-, tri-, and polyfunctional (meth)acrylates, graft cross-linkers having at least two different reactive C—C double bonds, for example alkylmethacrylates and alkylacrylates, as well as aromatic cross-linkers such as 1,2-divinylbenzene, 1,3-divinylbenzene, and 1,4-divinylbenzene, are suitable for this purpose. Amongst the difunctional (meth)acrylates, in particular the (meth)acrylates of propandiol, butandiol, hexandiol, octandiol, nonandiol, decandiol and eicosandiol as well as the di(meth)acrylates of ethyleneglycol, triethyleneglycol, tetraethyleneglycol, dodecaethyleneglycol, tetradecaethyleneglycol, propyleneglycol, dipropyleneglycol, and tetradecapropyleneglycol, moreover glyceroldi(meth)acrylate, 2,2-bis[(gamma-methacryloxy-beta-oxypropoxy)-phenylpropane], bis-GMA, bisphenol-A-dimethacrylate, neopentylglycol-di(meth)acrylate, 2,2-dimethacryloxypoly-ethoxyphenyl)propane with 2 to 10 ethoxy groups per molecule as well as 1,2-bis(3-methacryloxy-2-hydroxypropoxy)butane shall be mentioned. Exemplary multifunctional (meth)acrylates include, e.g. di-, tri- and/or tetra(meth)acrylates such as 1,4-butandiol-dimethacrylate, ethyleneglycol-dimethacrylate as well as di- or trivinylic compounds such as divinylbenzene.

The content of said cross-linker molecules in the starting mixture of the bead polymer preferably is in the range from 0.1% by weight to 10% by weight, in particular in the range from 0.5% by weight to 5% by weight.

Suitable alkylmethacrylates for liquid component (A) are methyl-, ethyl-, n-propyl, i-propyl-, n-butyl-, t-butyl-, i-butyl-, benzyl- and furfurylmethacrylates or mixtures thereof. Of these, methyl-methacrylate is particularly preferred.

In an expedient embodiment, at least one cross-linker is present in liquid component (A) aside from at least one of the above-mentioned monofunctional alkylmethacrylate monomers. This can, for example, be multifunctional monomers, comonomers or multifunctional oligomers. Aside from di-, tri-, and polyfunctional (meth)acrylates, graft cross-linkers having at least two different reactive C—C double bonds, for example alkylmethacrylates and alkylacrylates, as well as aromatic cross-linkers such as 1,2-divinylbenzene, 1,3-divinylbenzene, and 1,4-divinylbenzene, are suitable for this purpose. Amongst the difunctional (meth)acrylates, in particular the (meth)acrylates of propandiol, butandiol, hexandiol, octandiol, nonandiol, decandiol and eicosandiol as well as the di(meth)acrylates of ethyleneglycol, triethyleneglycol, tetraethyleneglycol, dodecaethyleneglycol, tetradecaethyleneglycol, propyleneglycol, dipropyleneglycol and tetradecapropyleneglycol, moreover glycerol-di(meth)acrylate, 2,2-bis[(gamma-methacryloxy-beta-oxypropoxy)-phenylpropane], bis-GMA, bisphenol-A-dimethacrylate, neopentylglycol-di(meth)acrylate, 2,2-dimethacryloxypoly-ethoxyphenyl)propane with 2 to 10 ethoxy groups per molecule as well as 1,2-bis(3-methacryloxy-2-hydroxypropoxy)butane shall be mentioned. Exemplary multifunctional (meth)acrylates include, e.g. di-, tri- and/or tetra(meth)acrylates such as 1,4-butandiol-dimethacrylate, ethyleneglycol-dimethacrylate as well as di- or trivinylic compounds such as divinylbenzene. It is self-evident that mixtures of said cross-linker molecules can be used as well. Also particularly well-suited are multifunctional compounds, in particular di- and/or trifunctional compounds, which possess elastic properties and are therefore suitable for imparting flexible properties on denture materials that are obtained from denture starting materials.

Dimethacrylates such as 1,4-butandiol-dimethacrylate are to be mentioned in exemplary manner in this context. Said cross-linker molecules can be present in liquid component (A) in amounts in the range from 0.1 to 20% by weight, preferably in the range from 1 to 10% by weight, for example 5% by weight.

In another embodiment, liquid component (A) may contain further comonomers aside from, e.g., methyl-methacrylate as the preferred main monomer (>50% by weight).

The radical initiator system required for polymerisation is contained in liquid component (A) and/or powder-shaped component (B) depending on reaction conditions and/or polymerisation system. Pertinent details are known to the person skilled in the art. For example in base mixtures for cold-polymers, the initiator system is most often present in both components, the liquid component and the powder-shaped component, and is thus combined when said components are mixed. Accordingly, one initiator component (C) is usually present in powder-shaped component (B), in particular in the form of peroxides, perketals, peresters and/or azo compounds. Another part of the initiator system (C), usually a co-initiator, can be present in liquid component (A). It is also feasible to use as initiators residual contents of initiator components that did not react during production of the powder-shaped components, e.g. peroxides such as dibenzoyl peroxide.

Conceivable initiators for the polymerisation reaction of cold- and/or auto-polymerising starting mixtures, as a matter of principle, are those that can be used to initiate radical polymerisation reactions. Peroxides and azo compounds are preferred initiators, for example the following:

### LPO: Dilauroylperoxid,

### BPO: Dibenzoylperoxide,

t-BPEH: tert.-Butylper-2-ethylhexanoate,

### AIBN: 2,2′-Azobis-(isobutyronitrile),

DTBP: Di-tert.-butylperoxide.

Suitable activators, e.g. aromatic amines, can be added to accelerate the radical polymerisation through peroxides. N,N-Dimethyl-p-toluidine, N,N-dihydroxyethyl-p-toluidine, and p-dibenzylamino-benzoic acid diethylester are exemplary as suitable amines. In this context, the amines usually function as co-initiators and are usually present in amounts of up to 0.5% by weight.

Moreover, redox systems, in particular combinations of dibenzoylperoxide, dilauroyl- or camphorquinone and amines such as N,N-dimethyl-p-toluidine, N,N-dihydroxyethyl-p-toluidine, and p-dimethylaminobenzoic acid diethylester, are suitable as radical initiator systems. Furthermore, it is also feasible to also use as redox systems those that also contain, aside from a peroxide, ascorbic acid or derivatives thereof, barbituric acid or a barbituric acid derivative or a sulfinic acid as reduction agent. In a preferred embodiment, a redox system of this type contains barbituric acid or thiobarbituric acid or a barbituric acid or thiobarbituric acid derivative (for example 25 to 80% by weight), at least one copper salt or one copper complex (for example 0.1 to 8% by weight), and at least one compound having an ionogenic halogen atom (for example 0.05 to 7% by weight). Exemplary suitable ingredients of the redox system mentioned above are 1-benzyl-5-phenylbarbituric acid, copper acetylacetonate, and benzyldibutylammoniumchloride.

Curing of the compositions preferably proceeds through redox-induced radical polymerisation at room temperature and/or at slightly elevated temperature and under a slight pressure in order to avoid the formation of bubbles. For example redox initiator combinations such as, e.g., combinations of benzoyl- or laurylperoxide and N,N-dimethyl-sym.-xylidine or N,N-dimethyl-p-toluidine, are used as initiators for the polymerisation that is carried out at room temperature.

Component (C) preferably contains barbituric acid or a barbituric acid derivative.

A combination of barbituric acids in conjunction with copper and chloride ions as well as above-mentioned peroxides is a particularly preferred initiator system. Said system is characterised by its high level of colour-fastness.

Moreover, powder-shaped component (B) and/or liquid component (A) can be provided in known manner with further additives from the group of stabilisers, UV-absorbing agents, thixotroping agents, and filling agents.

Moreover, powder-shaped component (B) and/or liquid component (A) can be provided with further additives, for example antimicrobial additives. Said suitable additives with a biocidal effect can comprise, e.g., in particular in the form of a mixture, at least one inorganic copper salt, at least one inorganic silver salt, and at least one inorganic barium salt as well as silver as an option. In this context, it is preferable for the copper salt to be copper sulfate, the silver salt to be silver phosphate, and the barium salt to be barium sulfate. According to another preferred embodiment, the invention provides the inorganic copper salt, in particular copper sulfate, and/or the inorganic silver salt, in particular silver phosphate, and/or the inorganic barium salt, in particular barium sulfate, and/or silver, if applicable, to be present on an inert substrate material. Preferably, the inorganic copper salt, in particular copper sulfate, and/or the inorganic silver salt, in particular silver phosphate, and/or the inorganic barium salt, in particular barium sulfate, as well as silver, if applicable, all are present on a substrate material. Suitable inert substrate materials comprise, e.g., polymer materials that are present in particulate form and also inorganic substances, e.g. silicates.

The material according to the invention is particularly well-suited as denture base material, but is also applied in veterinary medicine for hoof repair materials, for bone cement for cementing artificial articular prostheses, and for orthodontic appliances.

The invention is illustrated in more detail by the following examples without meaning to thus limit the invention. As is the case in the remaining description, specification of parts and percentages refer to the weight unless specified otherwise.

### Exemplary Embodiment 1a

Producing a Powder Mixture (A):

A mixture is produced from PMMA beads of different grain sizes and added barbituric acid.

### Exemplary Embodiment 1b

Producing a Liquid/Monomer Mixture According to the Invention (B):

A mixture is produced from methylmethacrylate, butandiol-dimethacrylate and another methacrylate-based cross-linker and added stabilisers and initiators (barbituric acid/copper system). In addition, the aliphatic urethane diacrylate according to the invention is added, i.e. in the present case Albiflex® XP6/1166 made by Nano Resins (Geesthacht, Germany).

### Exemplary Embodiment 2

Production of Test Bodies, Boiling Test, Determination of Colour Values, Determination of Mechanical Properties:

Powder mixture and monomer mixture at a ratio of 10:7 are mixed vigorously and test bodies with dimensions of 30×30×3 mm are poured after the swelling phase.

Test bodies are poured as reference 2 from a monomer mixture, which does not contain aliphatic urethane diacrylate, and the same powder. Moreover, test bodies are produced as reference 1 with a butadiene-acrylonitrile oligomer as additive (cf. EP1923037A2).

The test bodies are first measured by colorimetry (device: Datacolor® SF 600 spectrophotometer) and then stored in fully desalted water for 8 weeks at 50° C. Subsequently, the colour is measured again and the change of the b value and E value is determined. The monomer mixture according to the invention and the reference mixtures are also used to produce material for determination of flexural strength, e modulus, and fracture toughness. The results are summarised in the following tables.

| | | | | | Delta b | Delta E |
|---|---|---|---|---|---|---|
| | | | | Fracture | (Δyellow value) | (Δtotal colour |
| | Flexural | E | Fracture | intensity | after 8 weeks | change) after 8 |
| | strength | modulus | work | factor | of storage | weeks of storage |
| | [MPa] | [MPa] | [J/m²] | [MPa*m^{1/2}] | in water at 50° C. | in water at 50° C. |
| Monomer mixture | 70 | 2097 | 457 | 1.99 | 0.28 | 0.44 |
| according to the | | | | | | |
| invention | | | | | | |
| Reference 1 (with | 72 | 2160 | 642 | 2.33 | 5.42 | 5.68 |
| butadiene- | | | | | | |
| acrylonitril oligomer | | | | | | |
| additive) | | | | | | |
| Reference 2 | 75 | 2346 | 274 | 1.71 | 0.35 | 0.36 |
| (without additive) | | | | | | |

Explanation of Values:

Reference 2 without additive shows a low discolouration tendency, but also no improved fracture resistance.

Mixtures containing a plasticising additive for high fracture resistance (reference 1) tend to show yellowing and discolouration, though.

The monomer mixture according to the invention surprisingly shows both improved fracture toughness and lesser discolouration tendency. It is therefore very well-suited as denture base material.

1. Denture base material, obtained from an auto-polymerizing or cold-polymerizing composition that contains
(A) a liquid monomer component;
(B) a powder-shaped component;
(C) at least one initiator or one initiator system for auto- or cold-polymerization, wherein at least one member of the group consisting of aliphatic urethane acrylates and aliphatic urethane methacrylates is present in components (A) or/and (B).

2. Denture base material according to claim 1, wherein at least one liquid aliphatic urethane diacrylate resin composed of aliphatic urethane and acrylic segments is present in component (A), and the composition is free of plasticizing synthetic rubber.

3. Denture base material according to claim 1, having a fracture toughness of >1.8 MPa*m^{1/2} and a fracture work of >400 J/m² after curing.

4. Denture base material according to claim 1, which shows a difference of maximally one unit in the yellow value, also called b value, after 8 weeks of storage in fully desalted water at 50° C.

5. Denture base material according to claim 1, wherein the fraction of aliphatic urethane acrylate and/or urethane methacrylate is 0.5 to 20% by weight.

6. Denture base material according to claim 5, wherein the fraction of aliphatic urethane acrylate and/or urethane methacrylate is 0.1 to 5% by weight.

7. Denture base material according to claim 1, wherein the aliphatic urethane acrylate and/or urethane methacrylate has aliphatic urethane segments and (meth)acrylate segments in the polymer structure.

8. Denture base material according to claim 1, wherein the aliphatic urethane acrylate and/or urethane methacrylate is colorless and transparent.

9. Denture base material according to claim 1, wherein the aliphatic urethane acrylate and/or urethane methacrylate has no inherent odour.

10. Denture base material according to claim 1, obtained from an auto-polymerizing or cold-polymerizing composition containing components (A), (B), and (C), which, in addition, contains one or more substances selected from the group consisting of filling agents, pigments, stabilisers, modifiers, antimicrobial additives, UV-absorbing agents, thixotroping agents, catalysts, further monomers, and cross-linkers.

11. Hoof repair materials bone cement for cementing artificial articular prostheses and orthodontic appliances comprising the denture base material of claim 1.

12. Modifying agent in compositions for auto- or cold-polymerising denture base material comprising a member of the group consisting of aliphatic urethane acrylates and aliphatic methacrylates.

13. Modifying agent according to claim 12, whereby the modifying agent is a liquid aliphatic urethane diacrylate resin composed of aliphatic urethane and acrylic segments.

14. Modifying agent according to claim 12, wherein the compositions are free of plasticizing synthetic rubber.

15. Modifying agent according to claim 12, wherein no adverse effect on the results of the sun test according to ISO 20795 occurs.
